(19) United States
(12) Patent Application Publication
(10) Pub. No.: US 2026/0294361 A1
(43) Pub. Date: Oct. 1, 2026

(54) SYSTEM AND METHOD FOR REAL-TIME PROCESSING OF PET DETECTOR HIT EVENTS

(71) Applicant: Canon Kabushiki Kaisha, Tokyo (JP)

(72) Inventor: John BAUMGART, Vernon Hills, IL (US)

(73) Assignee: Canon Kabushiki Kaisha, Tokyo (JP)

(21) Appl. No.: 19/093,349

(22) Filed: Mar. 28, 2025

(51) Int. Cl. G01T 1/29

(52) U.S. Cl. CPC G01T 1/2985

(57) ABSTRACT

A method of processing a plurality of events, the method comprising sorting the plurality of events by time; first combining a first event with one or more events subsequent to the first event in the sorted plurality of events to generate a first subseries of events; second combining a second event with one or more events subsequent to the second event in the sorted plurality of events to generate a second subseries of events; removing, from the second subseries, one or more events that are included in both the first subseries and the second subseries; and after performance of the removing step, third combining the second subseries with one or more events in the sorted plurality of events subsequent to a final event in the second subseries to generate a third subseries of events.

### BACKGROUND

### Technical Field

The present disclosure relates to integration of photodetector signals.

### Description of the Related Art

Positron emission tomography (PET) is a functional imaging modality that is capable of imaging biochemical processes in humans or animals through the use of radioactive tracers. In PET imaging, a tracer agent is introduced into the patient to be imaged via injection, inhalation, or ingestion. After administration, the physical and bio-molecular properties of the agent cause it to concentrate at specific locations in the patient's body. The actual spatial distribution of the agent, the intensity of the region of accumulation of the agent, and the kinetics of the process from administration to its eventual elimination are all factors that may have clinical significance.

The foregoing “Background” description is for the purpose of generally presenting the context of the disclosure. Work of the inventors, to the extent it is described in this background section, as well as aspects of the description which may not otherwise qualify as prior art at the time of filing, are neither expressly or impliedly admitted as prior art against the present disclosure.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

In one embodiment, the present disclosure is related to a method of sorting positron emission tomography (PET) photodetector events, the method comprising converting acquired photodetector signals into a list of photodetector events; sorting the list of photodetector events by time; first combining a first photodetector event with one or more subsequent photodetector events in the sorted list of photodetector events to generate a subseries of photodetector events when the one or more subsequent photodetector events are acquired within a spatial range of a photodetector associated with the first photodetector event and a temporal range of an acquisition time of the first photodetector event; repeating the combining step for each photodetector event in the sorted list of photodetector events; removing one or more initial photodetector events from each generated subseries of photodetector events when the one or more initial photodetector events overlap with photodetector events in other subseries of photodetector events; second combining a first subseries of photodetector events and a second subseries of photodetector events when photodetector events in the first subseries are within the temporal range of photodetector events in the second subseries; and repeating the removing step and the second combining step for a predetermined number of iterations to generate one or more singles of photodetector events.

In one embodiment, the present disclosure is related to a positron emission tomography (PET) apparatus, comprising processing circuitry configured to: convert acquired photodetector signals into a list of photodetector events; sort the list of photodetector events by time; first combine a first photodetector event with one or more subsequent photodetector events in the sorted list of photodetector events to generate a subseries of photodetector events when the one or more subsequent photodetector events are acquired within a spatial range of a photodetector associated with the first photodetector event and a temporal range of an acquisition time of the first photodetector event; repeat the combining step for each photodetector event in the sorted list of photodetector events; remove one or more initial photodetector events from each generated subseries of photodetector events when the one or more initial photodetector events overlap with photodetector events in other subseries of photodetector events; combine a first subseries of photodetector events and a second subseries of photodetector events when photodetector events in the first subseries are within the temporal range of photodetector events in the second subseries; and repeat the removing step and the second combining step for a predetermined number of iterations to generate one or more singles of photodetector events.

In one embodiment, the present disclosure is related to a non-transitory computer-readable storage medium for storing computer readable instructions that, when executed by a computer, cause the computer to perform a method, the method comprising: converting acquired photodetector signals into a list of photodetector events; sorting the list of photodetector events by time; first combining a first photodetector event with one or more subsequent photodetector events in the sorted list of photodetector events to generate a subseries of photodetector events when the one or more subsequent photodetector events are acquired within a spatial range of a photodetector associated with the first photodetector event and a temporal range of an acquisition time of the first photodetector event; repeating the combining step for each photodetector event in the sorted list of photodetector events; removing one or more initial photodetector events from each generated subseries of photodetector events when the one or more initial photodetector events overlap with photodetector events in other subseries of photodetector events; combining a first subseries of photodetector events and a second subseries of photodetector events when photodetector events in the first subseries are within the temporal range of photodetector events in the second subseries; and repeating the removing step and the second combining step for a predetermined number of iterations to generate one or more singles of photodetector events.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a method of generating singles of photodetector events, according to one embodiment of the present disclosure;

FIG. 2 is a schematic of sorting photodetector events, according to one embodiment of the present disclosure;

FIG. 3A is a schematic of singles, according to one embodiment of the present disclosure;

FIG. 3B is a schematic of singles, according to one embodiment of the present disclosure;

FIG. 3C is a schematic of singles, according to one embodiment of the present disclosure;

FIG. 3D is a schematic of singles, according to one embodiment of the present disclosure;

FIG. 4 is a table of sorting times, according to one embodiment of the present disclosure;

FIG. 5 is a table of processing times, according to one embodiment of the present disclosure;

FIG. 6A shows a perspective view of a PET scanner that can be used with the techniques described herein, according to one embodiment of the present disclosure; and

FIG. 6B shows a schematic view of a PET scanner that can be used with the techniques described herein, according to one embodiment of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed. Further, spatially relative terms, such as “top,” “bottom,” “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The system may be otherwise oriented (rotated 90 degrees or at other orientations) and the spatially relative descriptors used herein may likewise be interpreted accordingly.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

In one embodiment, the present disclosure is directed to assembling singles of events generated by photodetectors in a positron emission tomography (PET) system. Photodetectors (or photosensors) in a PET system can generate an electrical output signal based on gamma ray interactions (hits) with the detector crystals. In one embodiment, the output signal can be a current pulse that is generated in response to a detected gamma photon. An annihilation event can produce two gamma photons, and each gamma photon can be detected by one or more photodetectors. One or more photodetector hits (photodetector events) that correspond to a single gamma photon can be grouped via a processing method into a single. The single can comprise hits that are temporally and spatially related and therefore are likely to result from a single photon's interaction with the photodetectors.

Hits can be temporally related when they occur within the same time window. Hits can be spatially related when they occur at photodetectors within the same region of photodetectors in the PET system. For example, photodetectors can be arranged in a sensor array. Hits to adjacent sensors or hits that occur across multiple sensors can be grouped into a single based on their spatial proximity. The ordering and grouping of hits into singles is useful for identifying an annihilation event and for identifying coincidence pairs of events that correspond to the same annihilation event. Singles can be indicative of a patient's anatomy for image reconstruction from the PET data.

A photodetector event can include spatial data in the form of an encoding of a photodetector that generates the photodetector event. Photodetector events can be received in a linear and highly ordered list. However, the photodetector events in the sequence may not be received in the same chronological order as when they occur because of the large number of events that can occur at a high frequency in a single PET scan. Customized hardware can be developed to receive and process photodetector events. However, hardware is inherently static and cannot be scaled up or down after installation or modified for algorithmic changes. Development and production of customized hardware is also expensive.

The present disclosure is directed to systems and methods for sorting and grouping photodetector events in order to assemble singles using a parallel computing environment. In one embodiment, the methods described herein can be implemented on one or more graphics processing unit (GPU) cores. The method can take advantage of multithreading capacities in a GPU core to process a list of events using multiple threads. Each thread can process the list in parallel passes, resulting in simultaneous and faster grouping of events throughout the list. Each thread in a process can access a shared memory and can write to the shared memory during the parallel processing. In one embodiment, each thread can access a list of events stored in memory to assemble singles from the events. In one embodiment, the methods can be pipelined over multiple GPUs to achieve higher throughputs of event processing. In one embodiment, the methods can be implemented in a hardware (e.g., field programmable gate array (FPGA)) configuration.

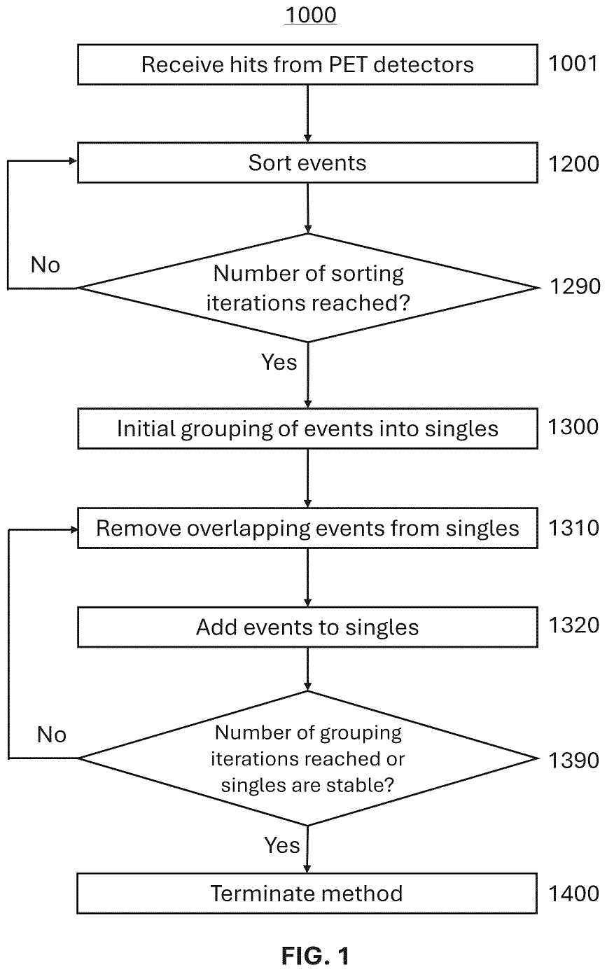

FIG. 1 is a flowchart of a method 1000 of assembling singles according to one embodiment. In step 1001, a batch of photodetector signals can be received and can be decoded into photodetector events. Each photodetector event can correspond to an output signal from a photodetector in the PET system. The photodetector events can be strictly ordered in a list because they are received in a first-in, first-out (FIFO) queue from the photodetectors. However, given that the photodetectors are spatially distributed in the PET system, it is possible that the order of events in the queue is not a strictly chronological order. In one embodiment, the FIFO queue can be received from the photodetectors via remote data memory access (RDMA), e.g., over peripheral component interconnect express (PCIe), Ethernet, etc. In one embodiment, the completion of the transfer of the photodetector signals can trigger the remaining steps of the method. In step 1200, the photodetector events can be sorted temporally via one or more sorting iterations. The sorting step 1200 can be repeated until a number of sorting iterations is reached at 1290. When the number of sorting iterations has been reached at 1290, the method can proceed to step 1300. In step 1300, each event in the sorted list can be assigned to a thread, and each thread can assemble one single by searching forward and adding spatially and temporally related events to the assigned event to form a single. Each event in the list is therefore the initial event in a single of subsequent events. The single of each thread can be considered a subseries of photodetector events that can be refined in further steps to generate a finalized single of photodetector events.

In step 1310, each thread can perform a backwards pass through its own single and can remove events that are part of singles in other threads corresponding to earlier events. Step 1310 removes overlap between singles across different threads. As a result, the initial event of each thread's single can be different (updated) after step 1310. In step 1320, each thread can perform a forward pass from the updated initial event of its single through singles of other threads and can combine singles that are temporally similar. A combined single can be processed on the thread corresponding to the earliest initial event of the singles that are combined to form the combined single.

In one embodiment, steps 1310 and 1320 can be repeated until a number of grouping iterations is reached. In one embodiment, the number of grouping iterations can be four. Each iteration can remove overlaps and combine or recombine singles to properly group the events based on their temporal and spatial origins. Repeating steps 1310 and 1320 can ensure that the singles are of a maximum length of related events while preventing overlap between singles. When the number of grouping iterations has been reached or the singles are stable at 1390, the method 1000 can terminate at step 1400.

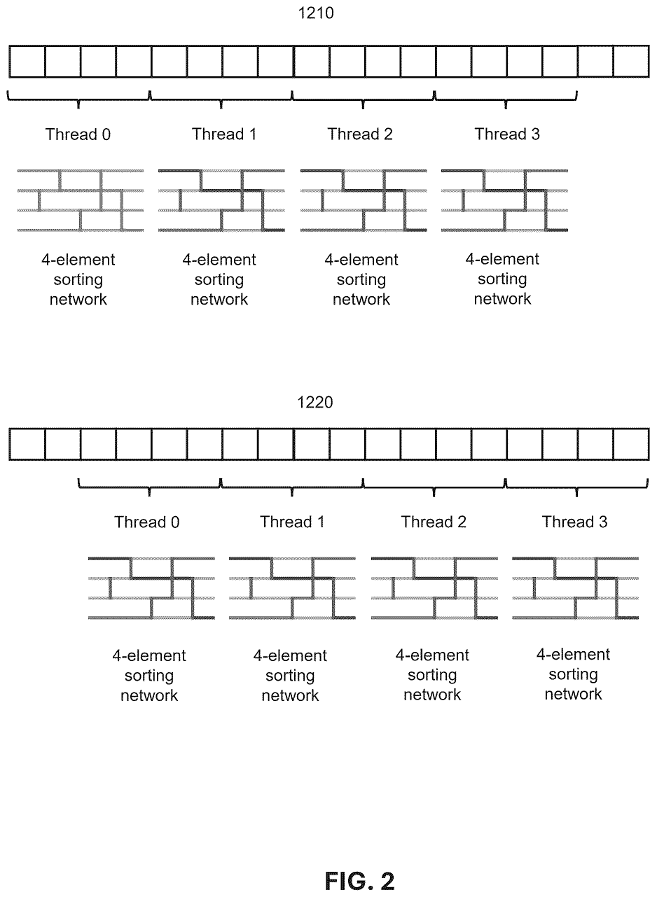

FIG. 2 is an illustration of the sorting step 1200 according to one embodiment. In one embodiment, the sorting can be a multithreaded process. A computing core (e.g., GPU core) can sort a list of photodetector events using sorting networks. In one embodiment, each thread can sort a number of events in the list using a sorting network. In one embodiment, the sorting step 1200 can include a first sorting pass 1210 and a second sorting pass 1220. The sorting step 1200 can include a number of iterations of the first and second sorting passes.

For example, FIG. 2 illustrates a first pass 1210 wherein each thread can sort a first group of four events in a first window using a four-element sorting network. Each thread's window of events can be distinct from another thread's window without overlap. A sorting network can compare events in pairs and swap events based on the pair comparisons until the events are ordered temporally. The maximum number of comparisons that a sorting network makes is dependent on the number of elements that are being sorted. In one embodiment, each thread can write the sorted group of events to an output buffer so that the sorted group of events can be read from memory by another thread in the second pass.

In the second pass 1220, each thread can sort a second group of four events in a second window using a four-element sorting network. In one embodiment, the second windows can overlap with the first window. For example, the second group of events can share half of its events with the first group of events. The embodiment of FIG. 2 illustrates a second group of four events for each thread in the second pass 1220, wherein the first two events in the second group for each thread are the latter two events in the first group in the first pass 1210.

In one embodiment, the first pass 1210 and the second pass 1220 can be repeated in sequence for each iteration. A thread can sort events in a first window followed by events in a second window in each iteration. It can be appreciated that the events within each window can be different after each sorting iteration. The list is iteratively sorted as each pass moves earlier events towards the beginning of the list and later events towards the end of the list. In one embodiment, for each step, the group of events to be sorted are received by a thread through an input buffer. After the group of events is sorted, the thread can swap the input buffer and the output buffer to output the sorted events for other threads to access and receive the next group of events to sort.

In one embodiment, the first pass 1210 and the second pass 1220 can be repeated for a number of sorting iterations. In one embodiment, the number of sorting iterations can be approximately 80 iterations. The initial ordering of the list may already correspond to a rough chronological order based on when the events are received from the photodetectors. As a result, it is not necessary to compare each event in the list to every other event in the list in order to sort the list chronologically. The sorting can be approximately localized based on the sizes of the groups and the number of sorting iterations. Limiting the number of iterations can limit the computational burden and complexity of the sorting process, while the overlapping sorting can leverage the ordering of the events to still generate a sufficiently sorted list. The runtime of the sorting step 1200 can therefore be constant.

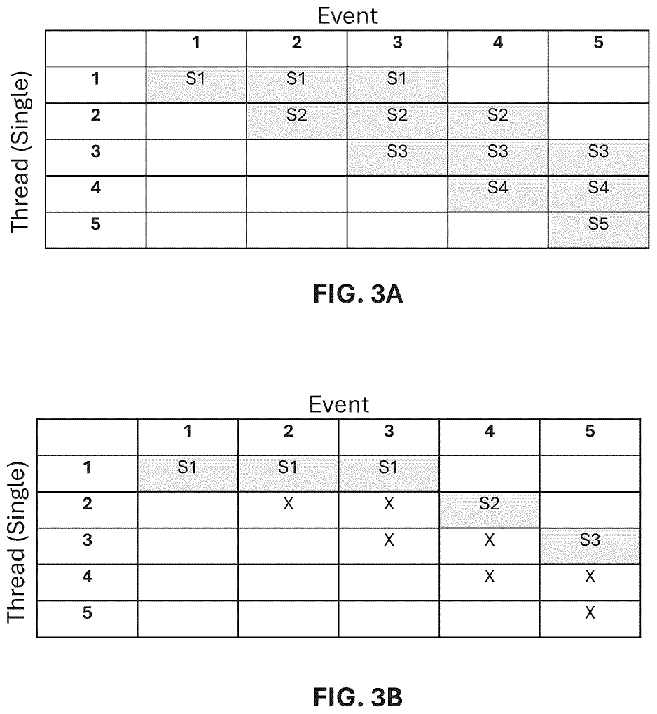

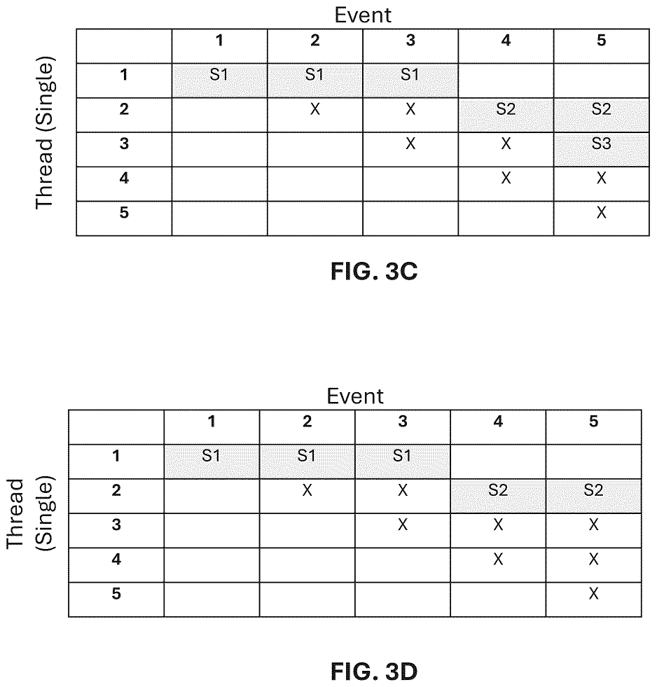

FIG. 3A illustrates the initialization of singles in the first grouping step 1300 (“initialization step”). After the list is sorted in step 1200, each event in the list can be assigned as an initial event to a thread in a multithreaded processor. For example, a first thread can be assigned a first event as an initial event, a second thread can be assigned a second event as an initial event, a third thread can be assigned a third event as an initial event, etc. In step 1300, each thread can perform a forward scan to access events that are subsequent to the initial event in the ordered list. Each thread can combine subsequent events into a single when the subsequent events are both spatially and temporally related to the initial event. In one embodiment, the forward scan can be limited to a temporal window. For example, a thread can access subsequent events until it reaches events that are outside of a temporal window from the initial event of the thread. Events outside of the temporal window do not need to be processed because they would not be in the same single as the initial event.

In FIG. 3A, the first thread can group the first event, second event, and third event into a single S1 because the second event and the third event are spatially and temporally related to the first event. The second thread can group the second event, third event, and fourth event into a single S2 because the third event and the fourth event are spatially and temporally related to the second event. The third thread can group the third event, the fourth event, and the fifth event into a single S3. The fourth thread can group the fourth event and the fifth event into a single S4. The fifth thread can group the fifth event into a single S5.

In one embodiment, events can be spatially related when they are generated by adjacent photodetectors. In one embodiment, events can be spatially related when they are generated by photodetectors that fall within a spatial region or area. The spatial region can be, for example, a number of photodetectors. In one embodiment, events can be temporally related when they fall within a given temporal window. The temporal window can be set based on the clock resolution of the PET detector. As an example, the temporal window can be on a nanosecond to picosecond scale. It is possible that adjacent events are not part of the same single because they are not spatially and temporally related. For example, a first event and a second event following the first event can fall within the given temporal window. However, the second event may originate from a photodetector that is not adjacent to the photodetector that generated first event; therefore, the first and second event cannot be added to the same single. In another example, the first and the second event can be generated by adjacent photodetectors but may not be in the same single because they were not generated within the given temporal window.

FIG. 3A illustrates five singles generated by five threads in step 1300. The threads can operate in parallel to build their respective singles by making a forward pass through the ordered list of events. Each single can comprise at least an initial event and a terminal event. In one embodiment, the threads can write to a shared memory to mark or indicate the events that have been combined into singles. It is possible that an event is stored in more than one single. For example, the third event of FIG. 3A is included in a first single of the first thread, a second single of the second thread, and a third single of the third thread. However, overlap between singles may frustrate image reconstruction because it is more difficult to identify coincidence events across different singles.

Therefore, a second grouping step 1310 (“reduction step”) can remove overlapping events from singles. In step 1310, each thread can identify overlapping events between other threads. Each thread can perform a backwards pass or scan of events in its single to identify events that are also grouped in other singles. For example, Thread 2 of FIG. 3A generates a single S2 that includes Event 2 as an initial event and Event 4 as a terminal event. Thread 2 can access singles generated by threads that have earlier initial events (e.g., Thread 1) and can identify whether Events 2-4 are included in the singles of those threads. Thread 2 can then remove overlapping events (e.g., Events 2 and 3) from S2.

FIG. 3B illustrates the threads of FIG. 3A after the second grouping step 1310. The overlapping events are removed from each single. In the example of FIG. 3B, the overlapping events that are removed for S2-S5 include the initial events of each single. However, the initial events do not necessarily overlap with other singles given that adjacent events may not be added to the same single when they are not spatially and temporally related.

In one embodiment, each thread can identify and remove overlapping events in parallel to reduce the time that it takes to complete the second grouping step 1310. Each thread can perform the process of identifying and removing overlapping events independently without inter-thread communication. As a result, the same event(s) can be removed from multiple threads, as illustrated in FIG. 3B. For example, S2 only has one event, Event 4, after step 1310. It is unlikely that a single only comprises one event; therefore, a corrective step can be implemented to combine singles that are too short with other events that are spatially and temporally related.

A third grouping step 1320 (“expansion step”) can be a corrective step wherein events are added to singles in a forward pass. In step 1320, each thread can access the sorted list of events and can add events that are spatially and temporally related to the earliest event of the thread's single. The earliest event in a thread's single can refer to the earliest remaining non-overlapping event after step 1310. The earliest event may or may not be the initial event assigned to the thread. The criteria for adding an event to a single can be the same in step 1320 as in step 1300. In one embodiment, the forward scan in step 1320 can also be limited to a temporal window. For example, a thread can access subsequent events until it reaches events that are outside of a temporal window from the earliest event of the thread. Events outside of the temporal window do not need to be processed because they would not be in the same single as the earliest event.

FIG. 3C illustrates the threads of FIG. 3B after the third grouping step 1320. Event 4 and Event 5 are combined in the S2 because they are spatially and temporally related. The third grouping step enables the threads to continue building singles after removing overlapping events. In one embodiment, the singles can be limited to a maximum number of elements. The maximum number of elements can be a predetermined number that can be set based on the PET system, allowing for flexibility in the grouping process.

Adding events to a single in the third grouping step 1320 can result in new overlap between singles. For example, FIG. 3C illustrates that Event 5 was added to S2, and S2 now overlaps with S3. In one embodiment, the method can include repeating grouping steps 1310 and 1320 in order to remove overlap and continue adding to singles. FIG. 3D illustrates an example of the events after repeating steps 1310 and 1320. As illustrated in FIG. 3D, repeating grouping step 1310 with the singles of FIG. 3C results in overlapping Event 5 being removed from S3. The events pictured in FIG. 3D are then grouped into two distinct singles, the first single S1 comprising Events 1-3 and the second single S2 comprising (at least) Events 4-5. The singles are non-overlapping and are comprehensive groupings of all events that are spatially and temporally related.

Repeating the grouping steps 1310 and 1320 can increase the size of singles within the size limit while ensuring that the singles only include events that are spatially and temporally related. The backwards pass of step 1310 removes unnecessary overlapping events from singles. The threads can then add events to their singles in the second forward pass 1320 without being constrained by the unnecessary overlapping events. Without the combination of the two steps, the singles may overlap and/or may not capture all spatially and temporally related events that result from a photon interaction.

In one embodiment, the grouping steps 1310 and 1320 can be repeated for a predetermined number of grouping iterations. The number of grouping iterations can be set based on the PET system, allowing for flexibility in the grouping process. In one embodiment, the number of grouping iterations can be related to the maximum number of events per single. For example, as the maximum number of events increases, the number of iterations can increase to process larger overlaps and additions in each iteration.

In one embodiment, the grouping steps 1310 and 1320 can be repeated until the singles are stable and do not change with additional passes through the element. In one embodiment, when the stability condition is met, the grouping process can be terminated with a final reduction step to ensure that there is no overlap between the singles.

In one embodiment, each thread can maintain input and output buffers to avoid data dependencies and keep data that is read (input) and data that is written (output) separate. In one embodiment, the threads do not perform read-modify-write operations while performing the parallel passes in steps 1310 and 1320. In one embodiment, the input and the output buffers can be swapped after each iteration to update the list of events. In one embodiment, the threads can synchronize after each pass (step). In one embodiment, a thread can further include a processing buffer. In one embodiment, the threads can be configured with one or more CUDA streams to hide data transfer times.

In one embodiment, the method can include providing a completion progress update of the assembly method. For example, the method can include a total number of steps including a number of sorting iterations and a number of grouping iterations. In one embodiment, the number of sorting iterations and/or the number of grouping iterations can be predetermined numbers. In one embodiment, the number of sorting iterations and/or the number of grouping iterations can be received as an input (e.g., a user input). In one embodiment, the PET detector system can be configured to display or otherwise indicate a progress or status of the assembly method. For example, the progress of the assembly method can be indicated as a percentage of the total steps that have been completed. The progress can be updated as the method progresses.

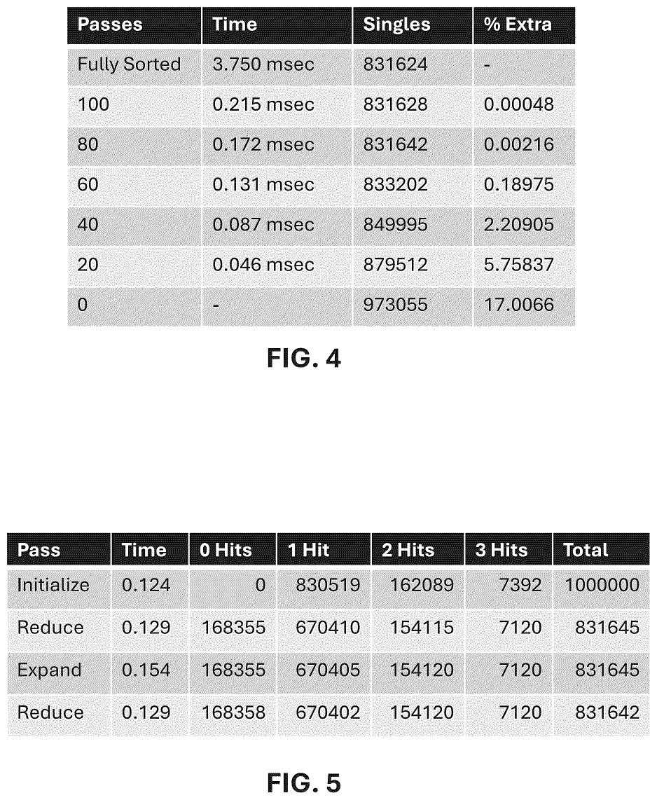

The method 1000 can improve the efficiency of single construction compared to conventional methods. The sorting step 1200 and the grouping steps 1300-1320 can both contribute to faster runtimes. As an example, the sorting step 1200 using 80 sorting iterations can be performed in 0.172 ms (milliseconds) for 10⁶ events, while a conventional full sorting method can take 3.75 ms. FIG. 4 is a table of experimental results for varying number of sorting iterations for a sample of 10⁶ events. A limited number of sorting iterations (e.g., from 20 to 100 iterations) can sort the ordered list of events much more quickly than a full sort algorithm with a small number of leftover events (% Extra).

FIG. 5 is a table of experimental results for the grouping steps 1300-1320 for a sorted sample of 10⁶ events. The sample of 10⁶ events can be sorted using the sorting step 1200 with 80 iterations. The grouping process of FIG. 5 can include cycling between the reduction step 1310 and the expansion step 1320 until the singles are stable. As an example, the singles can be stable after 3 passes through steps 1300-1320. In one embodiment, the initialization step and subsequent expansion steps can take 0.124 ms, the reduction steps can take 0.129 ms each for a total of 0.258 ms, and the expansion steps can take 0.154 ms for a total assembly time of approximately 0.536 ms to group the 10⁶ events into singles. The assembly time is much faster than conventional methods, which can take approximately 992 ms for 10⁶ events using an optimized CPU or even with a combination of CPU and GPU.

In one embodiment, the total time for processing 10⁶ events using the methods described herein can be approximately 1.158 ms. The total processing time can include approximately 0.280 ms to decode the photodetector events, approximately 0.172 ms to sort the events, approximately 0.536 ms to assemble the singles, and approximately 0.250 ms of additional processing time, e.g., for data transfer. When the events are encoded in 16 bytes of data, the methods of the present disclosure can assemble singles with a throughput of approximately 13.8 gigabytes (GB)/sec. Conventional methods are much less efficient and have throughputs of 10-16 MB/sec.

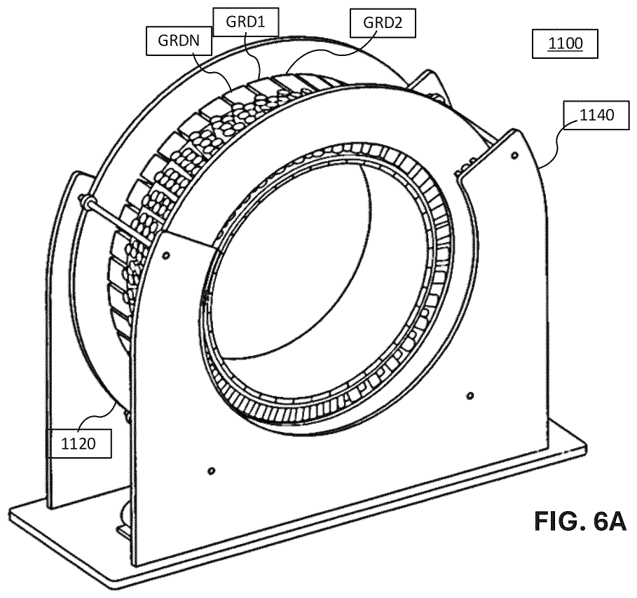

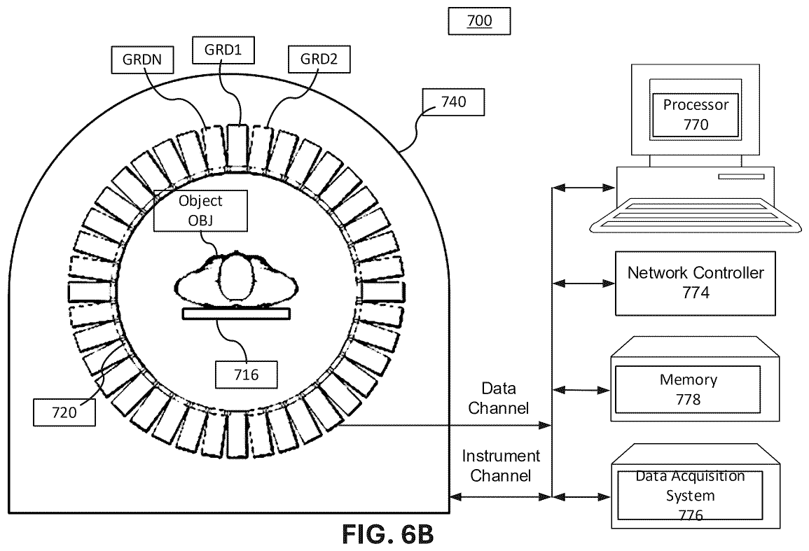

FIG. 6A and FIG. 6B show a non-limiting example of a PET scanner 700 that can implement the method 1000. The PET scanner 700 includes a number of gamma-ray detectors (GRDs) (e.g., GRD1, GRD2, through GRDN) that are each configured as rectangular detector modules. According to one implementation, the detector ring includes 40 GRDs. In another implementation, there are 48 GRDs, and the higher number of GRDs is used to create a larger bore size for the PET scanner 700.

Each GRD can include a two-dimensional array of individual detector crystals, which absorb gamma radiation and emit scintillation photons. The scintillation photons can be detected by a two-dimensional array of photomultiplier tubes (PMTs) that are also arranged in the GRD. A light guide can be disposed between the array of detector crystals and the PMTs.

Alternatively, the scintillation photons can be detected by an array of silicon photomultipliers (SiPMs), and each individual detector crystals can have a respective SiPM.

Each photodetector (e.g., PMT or SiPM) can produce an analog signal that indicates when scintillation events occur, and an energy of the gamma ray producing the detection event. Moreover, the photons emitted from one detector crystal can be detected by more than one photodetector, and, based on the analog signal produced at each photodetector, the detector crystal corresponding to the detection event can be determined using Anger logic and crystal decoding, for example.

FIG. 6B shows a schematic view of a PET scanner system having gamma-ray (gamma-ray) photon counting detectors (GRDs) arranged to detect gamma-rays emitted from an object OBJ. The GRDs can measure the timing, position, and energy corresponding to each gamma-ray detection. In one implementation, the gamma-ray detectors are arranged in a ring, as shown in FIG. 6A and FIG. 6B. The detector crystals can be scintillator crystals, which have individual scintillator elements arranged in a two-dimensional array and the scintillator elements can be any known scintillating material. The PMTs can be arranged such that light from each scintillator element is detected by multiple PMTs to enable Anger arithmetic and crystal decoding of scintillation event.

FIG. 6B shows an example of the arrangement of the PET scanner 700, in which the object OBJ to be imaged rests on a table 716 and the GRD modules GRD1 through GRDN are arranged circumferentially around the object OBJ and the table 716. The GRDs can be fixedly connected to a circular component 720 that is fixedly connected to the gantry 740. The gantry 740 houses many parts of the PET imager. The gantry 740 of the PET imager also includes an open aperture through which the object OBJ and the table 716 can pass, and gamma-rays emitted in opposite directions from the object OBJ due to an annihilation event can be detected by the GRDs and timing and energy information can be used to determine coincidences for gamma-ray pairs.

In FIG. 6B, circuitry and hardware are also shown for acquiring, storing, processing, and distributing gamma-ray detection data. The circuitry and hardware include: a processor 770, a network controller 774, a memory 778, and a data acquisition system (DAS) 776. The PET imager also includes a data channel that routes detection measurement results from the GRDs to the DAS 776, the processor 770, the memory 778, and the network controller 774. The DAS 776 can control the acquisition, digitization, and routing of the detection data from the detectors. In one implementation, the DAS 776 controls the movement of the bed 716. The processor 770 performs functions including reconstructing images from the detection data, pre-reconstruction processing of the detection data, and post-reconstruction processing of the image data, as discussed herein. The processor 770 can perform the determination of calibration parameters in the calibration phase and/or application of energy correction in the correction phase.

The processor 770 can be configured to perform various steps of the methods described herein and variations thereof. The processor 770 can include a CPU that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory may be non-volatile, such as ROM, EPROM, EEPROM or FLASH memory. The memory can also be volatile, such as static or dynamic RAM, and a processor, such as a microcontroller or microprocessor, may be provided to manage the electronic memory as well as the interaction between the FPGA or CPLD and the memory.

Alternatively, the CPU in the processor 770 can execute a computer program including a set of computer-readable instructions that perform various steps of the methods described herein, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xenon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

The memory 778 can be a hard disk drive, CD-ROM drive, DVD drive, FLASH drive, RAM, ROM or any other electronic storage known in the art.

The network controller 774, such as an Intel Ethernet PRO network interface card from Intel Corporation of America, can interface between the various parts of the PET imager. Additionally, the network controller 774 can also interface with an external network. As can be appreciated, the external network can be a public network, such as the Internet, or a private network such as an LAN or WAN network, or any combination thereof and can also include PSTN or ISDN sub-networks. The external network can also be wired, such as an Ethernet network, or can be wireless such as a cellular network including EDGE, 3G, 4G, and 5G wireless cellular systems. The wireless network can also be WiFi, Bluetooth, or any other wireless form of communication that is known.

The order of discussion of the different steps as described herein has been presented for clarity's sake. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

Obviously, numerous modifications and variations of the present disclosure are possible in light of the above teachings. It is therefore to be understood that within the scope of the appended claims, the invention may be practiced otherwise than as specifically described herein.

1. A method of processing a plurality of events, each of the plurality of events corresponding to a detection result of one or more photodetectors configured to detect gamma rays, the one or more photodetectors being mounted on a positron emission tomography (PET) apparatus, the method comprising:
sorting the plurality of events by time;
first combining a first event with one or more events subsequent to the first event in the sorted plurality of events to generate a first subseries of events, the one or more events subsequent to the first event being acquired within a first spatial range of a first photodetector associated with the first event and a first temporal range of an acquisition time of the first event;
second combining a second event with one or more events subsequent to the second event in the sorted plurality of events to generate a second subseries of events, the one or more events subsequent to the second event being acquired within a second spatial range of a second photodetector associated with the second event and a second temporal range of an acquisition time of the second event;
removing, from the second subseries, one or more events that are included in both the first subseries and the second subseries; and
after performance of the removing step, third combining the second subseries with one or more events in the sorted plurality of events subsequent to a final event in the second subseries to generate a third subseries of events, the one or more events subsequent to the final event being acquired within a third spatial range of a third photodetector associated with the final event and a third temporal range of an acquisition time of the final event.

2. The method of claim 1, wherein the first combining step and the second combining step are performed by parallel software threads.

3. The method of claim 1, wherein the first combining step further comprises combining the first event with the one or more events subsequent to the first event in the sorted plurality of events, the one or more events subsequent to the first event being acquired within the first temporal range based on a clock resolution of the first photodetector associated with the first event.

4. The method of claim 1, further comprising displaying a completion progress of the first combining, the second combining, the removing, and the third combining.

5. The method of claim 1, further comprising reconstructing an image based on the first subseries of events and the third subseries of events.

6. The method of claim 1, wherein the first subseries is before the second subseries in the sorted plurality of events and the second subseries is before the third subseries in the sorted plurality of events.

7. A positron emission tomography (PET) apparatus including one or more photodetectors configured to detect gamma rays, comprising:
processing circuitry configured to:
sort a plurality of events by time, each of the plurality of events corresponding to a detecting result of a photodetector of the one or more photodetectors;
first combine a first event with one or more events subsequent to the first event in the sorted plurality of events to generate a first subseries of events, the one or more events subsequent to the first event being acquired within a first spatial range of a first photodetector associated with the first event and a first temporal range of an acquisition time of the first event;
second combine a second event with one or more events subsequent to the second event in the sorted plurality of events to generate a second subseries of events, the one or more events subsequent to the second event being acquired within a second spatial range of a second photodetector associated with the second event and a second temporal range of an acquisition time of the second event;
remove, from the second subseries, one or more events that are included in both the first subseries and the second subseries; and
after the removing step, third combine the second subseries with one or more events in the sorted plurality of events subsequent to a final event in the second subseries to generate a third subseries of events, where the one or more events subsequent to the final event are being acquired within a third spatial range of a third photodetector associated with the final event and a third temporal range of an acquisition time of the third event.

8. The apparatus of claim 7, wherein the processing circuitry is further configured to combine the first event with the one or more events subsequent to the first event in the sorted plurality of events when the one or more events subsequent to the first event are acquired within the temporal range based on a clock resolution of the photodetector associated with the first event.

9. The apparatus of claim 7, wherein the processing circuitry is further configured to display a completion progress of the first combine, the second combine, the remove, and the third combine.
